(19) United States
(12) Patent Application Publication
(10) Pub. No.: US 2023/0414573 A1
(43) Pub. Date: Dec. 28, 2023

(54) SYNERGISTIC OPHTHALMOLOGICAL COMPOSITION IN A LOW-CONCENTRATION DOSE THAT IS EFFECTIVE IN THE PREVENTION, CONTROL AND ERADICATION OF PRESBYOPIA

(71) Applicant: Cesar Alejandro SÁNCHEZ GALEANA, CIUDAD DE MEXICO (MX)

(72) Inventor: Cesar Alejandro SÁNCHEZ GALEANA, CIUDAD DE MEXICO (MX)

(21) Appl. No.: 18/036,666

(22) PCT Filed: Nov. 11, 2021
(86) PCT No.: PCT/MX2021/050074

§ 371 Date: May 12, 2023

(30) Foreign Application Priority Data
Nov. 12, 2020 (MX) MX/A/2020/012116

(51) Int. Cl. A61K 31/4178; A61K 9/00; A61K 31/4174; A61K 31/498; A61K 31/728; A61K 31/196; A61K 45/06; A61P 27/10

(52) U.S. Cl. CPC A61K 31/4178; A61K 9/0048; A61K 31/4174; A61P 27/10; A61K 31/728; A61K 31/196; A61K 45/06; A61K 31/498

(57) ABSTRACT

An ophthalmic composition for the prevention, control and treatment of presbyopia symptoms comprising a synergistic combination of pilocarpine, brimonidine, oxymetazoline, hyaluronic acid, bromfenac and optionally a vasoconstrictor in a single low concentration dose to improve near vision ability and increase depth of focus is described. In addition, the side effects of pilocarpine administered separately, such as eye redness, headache, and dryness of the eye, are reduced and are better tolerated in patients suffering from presbyopia.

### FIELD OF THE INVENTION

The herein invention relates to the technical field of the pharmaceutical industry and concerns an ophthalmic composition comprising the synergistic combination of: one or more cholinergic agonist agents (pilocarpine) or their racemic forms or their pharmaceutically acceptable salts, one or more alpha adrenergic 1 agonist agents (oxymetazoline) or their racemic forms or their pharmaceutically acceptable salts, one or more alpha adrenergic 2 agonist agents (brimonidine) or their racemic forms or their pharmaceutically acceptable salts, one or more glycosaminoglucuronan agents (hyaluronic acid) or their racemic forms or pharmaceutically acceptable salts and one or more non-steroidal anti-inflammatory agents (bromfenac) or their racemic forms or pharmaceutically acceptable salts and/or one or more vasoconstrictor agents or their racemic forms or pharmaceutically acceptable salts; plus pharmaceutically acceptable vehicles, excipients or adjuvants, formulated in a pharmaceutical form of solution for ophthalmic administration. Said combination is indicated for the prevention, control and eradication of the symptoms of presbyopia, to increase the depth of focus in the eyes of patients suffering from presbyopia and to improve near vision ability.

The ophthalmologic composition produces a greater therapeutic effect when administered in a single dose, as opposed to when they are administered independently, generating benefits; lower dose administered, greater therapeutic effect and decreasing or eliminating adverse effects.

### BACKGROUNDS

Presbyopia is known to the refractive surgeon as the ultimate goal of correction. It is a great challenge, due to the complex mechanism the eye has to achieve accommodation, involving the crystalline lens and the ciliary body. Attempts at correction include: refraction: monovision with contact lenses, laser surgery: monovision, multifocal cornea, presbyopia; implants: Kamra, Raindrop, scleral expansion implants, and related lenses: femtosecond laser, pseudoaccommodative lenses: Crystalens; multifocal and pharmacologic intraocular lenses: parasympathetic mimetics such as pilocarpine, carbachol and aceclidine in various compounds.

In the ophthalmologic use, pilocarpine (belonging to an active substances class called cholinergic agonists) has been the most used active substance in the pharmacological treatment of presbyopia since over a hundred years.

Presbyopia is the physiological process by which human being losses its ability for visual perception of the close objects for accommodation deficit.

Typically, this deficit is attributed to decreased elasticity of the crystalline lens. Currently, a new theory assigns it to the progressive loss of ciliary muscle action.

The cholinergic agonist agents, so called due to the that acetylcholine is the neurotransmitter used by the parasympathetic system, its effects are similar to the produced by acetylcholine. Drugs in this family can act directly on the acetylcholine receptor, either nicotinic or muscarinic, either by inhibiting the cholinesterase enzyme by promoting the release of acetylcholine or by other indirect mechanisms. Among direct acting cholinergic agonists include: acetylcholine, bethanechol, carbachol, methacholine, nicotine, muscarine and pilocarpine, while indirectly acting cholinergic agonists include: donepezil, edrophonium, neostigmine, physostigmine, pyridostigmine, rivastigmine, tacrine, echothiophate, cisapride, metoclopramide, cionidine, propranolol, atenolol, prazosin, and methyldopa.

Pilocarpine is a mimetic parasympathetic drug binding to m3 muscarinic receptors. They are mainly used for the treatment of narrow-angle glaucoma in concentrations ranging from 1% to 4% causing miosis, contraction of the ciliary body increases uveoscleral outflow and then decreases intraocular pressure. Side effects include ciliary spasm, myopization, red eyes, eyebrow pain and posterior iris synechiae from chronic use.

Brominidine belongs to a class of active substances called alpha-adrenergic agonists that act by decreasing the amount of fluid present inside the eyes; and is indicated for the prevention or control of an increase in intraocular pressure (IOP), thus reducing elevated intraocular pressure by decreasing the degree of aqueous humor production, as well as by producing an increase in uveo-scleral flow.

Oxymetazoline is an imidazole derivative with a potent sympathomimetic effect that causes local vasoconstriction at the scleroconjunctival level. It is characterized by a rapid onset of action taking only a few minutes, a relatively long duration of action of approximately 6 or more hours and a low tendency to rebound congestion. Oxymetazoline is indicated for the symptomatic relief of conjunctival redness in mild ocular superficial irritations, due to any cause (allergies, irritant factors, ocular fatigue).

Hyaluronic acid or its sodium salt (HA) is a linear polymer of high molecular weight and natural origin; its monomeric unit is constituted by the disaccharide composed of N-acetyl-ß-D-glucosamine and ß-D-glucuronic acid.

HA is a vital component of ocular physiology that provides the viscosity required by the vitreous humor and keeps the corneal epithelium hydrated. HA's ability to be one of the eye's natural lubricants has been used to develop ophthalmic applications such as vitreous humor replacement, protection of the corneal endothelium against mechanical trauma during surgery and to mimic natural tears in the treatment of dry eye.

Bromfenac ophthalmic belongs to a class of active substances known as non-steroidal anti-inflammatory drugs (NSAIDs), which works to block the cyclo-oxygenase cycle that releases prostaglandins that cause pain or swelling. It is used to treat eye inflammation and redness (swelling) and pain that occurs after cataract surgery.

In the prior art, topical ophthalmic compositions are described which are mentioned below: patent application US2006177430 describes a sterile ophthalmic formulation for the treatment of an ophthalmic condition comprising methylsulfonylmethane and an amount of an ophthalmologically active agent effective for the treatment of that condition in a pharmaceutically acceptable vehicle, wherein the methylsulfonylmethane is present in an amount of at least about 1% by weight, and the pharmaceutically acceptable vehicle is at least partially aqueous, wherein the ophthalmologically active agent is an antioxidant, selected from vitamin A, vitamin C, vitamin E, lycopene, selenium, alpha-lipoic acid, coenzyme Q, glutathione or a carotenoid. It is also described that the ophthalmologically active agent is a metal complexing agent, a non-steroidal anti-inflammatory drug, an antibiotic, an antihistamine; wherein the ophthalmologically active agent is selected from aceclidine, acetazolamide, anecortave, apraclonidine, atropine, azapentacene, azelastine, bacitracin, befunolol, betamethasone, betaxolol, bimatoprost, brimonidine, brincoxolamidine, brincoxol, chlortetracycline, ciprofloxacin, cromoglycate, cromolyn, cyclopentolate, cyclosporine, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropin, fludrocortisone, fluorometholone, flurbiprofen, fomivirsen, framycetin, ganciclovir, gatifloxacin, gentamicin, homatropine, hydrocortisone, idoxuridine, indomethacin, isoflurophate, ketorolac, ketotifen, latanoprost, levobetaxololol, levobunolol, levocabastine, levofloxacin, lodoxamide, medidoprazole, moxacinol, methofluoride in, norfloxacin, ofloxacin, olopatadine, oxymetazoline, pemirolast, pegaptanib, phenylephrine, physostigmine, pilocarpine, pindolol, pyrenoxine, polymyxin B, prednisolone, proparacaine, ranibizumab, rimexolone, squalamine, tetrahydroamine, tetracyclamine, tetrizoline, timolol, tobramycin, travoprost, triamcinulone, trifluoromethasolamide, trifluridine, trimethoprim, tropicamide, unoprostone, vidarbine, xylometazoline, pharmaceutically acceptable salts thereof and combinations thereof. The ophthalmic condition is selected from macular degeneration, cataract, secondary cataract, glaucoma, elevated intraocular pressure, diabetic retinopathy, infection, allergy, pruritus, and inflammation.

The patent application MX/a/2013/003639 describes a method for treating dermatological conditions and symptoms associated therewith, comprising topically administering an effective amount of a combination of a combination of brimonidine or a pharmaceutically acceptable salt thereof and oxymetazoline or a pharmaceutically acceptable salt thereof, wherein the dermatological conditions do not include, and are not associated with rosacea; wherein the brimonidine is its tartrate salt and the oxymetazoline is hydrochloride. Dermatological conditions are selected from erythema, telangiectasia, actinic telangiectasia, psoriasis, skin cancer, pemphigus, sunburn, dermatitis, eczema, rashes, among other symptoms. Said composition comprises a pharmaceutically acceptable vehicle selected from lotions, gels, creams, ointments, pastes, emulsions, sprays, sprays, solutions, washes and shampoos.

Patent EP2645993 protects a pharmaceutical composition comprising oxymetazoline, ketostearyl alcohol, polyethylene glycol 6 stearate (PEG-6), polyethylene glycol 32 stearate (PEG-32), and glycol stearate, wherein the composition is a cream, provided that the cream does not have the following composition: brimonidine tartrate 0.5%, oxymetazoline hydrochloride 0.5%, phenoxyethanol 0.8%, methylparaben 0.2%, propylparaben 0.05%, disodium EDTA 0.01%, butylated hydroxytoluene 0.05%, PEG-300 4.0%, PEG-6 stearate (and) glycol stearate (and) PEG-32 stearate 7.5%, ketostearyl alcohol 4.0%, caprylic capric triglycerides 7.0%, diisopropyl adipate 7.0%, oleyl alcohol 7.0%, lanolin USP 2.0%, ceteareth-6 (and) stearyl alcohol 2.0%, ceteareth-25 2.0%, tartaric acid 0.001%, water DI 55.389%. The patent is focused on a composition comprising oxymetazoline, ketostearyl alcohol, polyethylene glycol 6 stearate (PEG-6), polyethylene glycol 32 stearate (PEG-32), and glycol stearate, wherein the composition is a cream, and wherein oxymetazoline is the only active pharmaceutical ingredient in the formulation and contains an emulsifier in an amount of 1% to 30% by weight, an emollient in an amount of 1% to 50% by weight, wherein the emulsifier and emollient are in a ratio of 0.7:1 to 1.8:1, wherein the emollient is selected from fatty esters, fatty alcohols or combinations thereof. It further comprises an additive selected from the group consisting of preservatives, emulsion stabilizers, pH adjusters, chelating agents, viscosity modifiers, antioxidants, surfactants, emollients, opacifying agents, skin conditioners, buffers. and combinations thereof, wherein the emollient is selected from fatty esters, fatty alcohols, or combinations thereof. The composition further comprises a topically active pharmaceutical agent selected from the group consisting of an antimycobacterial agent, an anti-rosaceous agent, and a mixture thereof, further comprising a vasoconstrictor. The patent description refers to which in some embodiments, the topically active cosmetic or pharmaceutical agent can include, without limitation, pilocarpine.

The International Patent Application WO2020144546 in its abstract refers to pharmaceutical compositions comprising a therapeutically effective amount of a selective alpha-adrenergic receptor agonist or anticholinergic agent or a pharmaceutically acceptable salt or stereoisomer thereof in combination with a therapeutically effective amount of lipoic acid or a pharmaceutically acceptable salt or stereoisomer thereof; wherein the selective alpha-adrenergic receptor agonist or anticholinergic agent is selected from the group consisting of pilocarpine, brimonidine and oxymetazoline, or a pharmaceutically acceptable salt or stereoisomer thereof, and their uses in the treatment or alleviation of xerostomia, dermal diseases and ocular disorders, however, said patent application seeks to protect pharmaceutical compositions comprising a physical mixture of: a therapeutically effective amount of a selective alpha-adrenergic receptor agonist or a pharmaceutically acceptable salt or stereoisomer, or an enantiomer thereof; and a therapeutically effective amount of lipoic acid or a pharmaceutically acceptable salt or stereoisomer or prodrug or an enantiomer thereof, wherein the lipoic acid is (R)-(-i-)-lipoic acid or lipoic acid or (R/S)-lipoic acid or preferably R-(+)-lipoic acid. wherein the composition is characterized in that the selective alpha-adrenergic receptor agonist agent is selected from a compound of Formula II selected from brimonidine tartrate and R-(+)-lipoic acid, it is also mentioned that the physical mixture comprises the compound of Formula III selected from oxymetazoline hydrochloride and R-(+)-lipoic acid, wherein the composition comprises at least one pharmaceutically acceptable excipient, said composition is formulated as drops, solution, emulsion, paste, gel, cream, ointments, spray, tablets, effervescent tablets, mucoadhesive formulation, subdermal, transdermal, hydrogel formulation, injections, sustained release for oral, ocular, dermal, parenteral, topical and nasal administration; which is used in the treatment or relief of xerostomia, burning mouth syndrome, dermal disorders and ocular diseases or disorders or a complication thereof.

Patent ES2725002 filed in year 2008 under the title “Medicament comprising pilocarpine and brimonidine”, which relates to topical compositions for improving visual acuity and methods for the use thereof. More particularly, but not exclusively, it refers to ophthalmic drops for improving the effects of presbyopia, myopia, hyperopia, astigmatism, and combinations thereof.

The drug of patent ES2725002 is adapted for topical administration to a human or animal eye, comprising at least two pharmacologically active agents, a first said active agent comprising a parasympathetic agonist, and a second said active agent comprising a sympathetic agonist. Said parasympathetic agonist comprises pilocarpine in an amount between about 0.05% and about 4% pilocarpine, optionally at least about 0.25%, and optionally not more than about 0.5%. The second active agent comprises a sympathetic agonist. Said sympathetic agonist comprises brimonidine in an amount between about 0.01% and about 4% brimonidine, optionally at least 40 about 0.1% thereof.

Patent ES2725002 a second eye drop formulation materializing the present invention was prepared incorporating 0.1% by weight of brimonidine and 0.25% by weight of pilocarpine in an otherwise conventional eye drop formulation. Brimonidine is classified as a sympathetic agonist; pilocarpine, as noted above, is considered a parasympathetic agonist. Three patients were evaluated with the second eye drop formulation In each case, administration of the second eye drop formulation produced effects of an improvement qualitatively in the patient's night vision, as the patient noticed fewer halos and glare, and quantitatively, from 6/6 to 6/5 in dim light conditions. These effects were maintained for at least two hours and some for at least four hours.

So in the patent family it was found that in Europe patent EP3031457 applied for in 2016, which has the same subject matter, was granted on Feb. 6, 2019 and the protected subject matter is as follows: The use of a combination of a first pharmacologically active agent comprising pilocarpine and a second pharmacologically active agent comprising brimonidine in the manufacture of a medicament adapted for topical application to a human or animal eye in order to improve visual acuity in one or more of presbyopia, myopia, hyperopia, emmetropia and astigmatism, and/or in order to improve night or low light vision. A use of a combination of pharmacologically active agents the medicament comprises at least 0.25% pilocarpine. A use of a combination of pharmacologically active agents characterized in that the medicament comprises between 0.05%, or 0.1%, brimonidine and at least 0.5% or 4% pilocarpine. A use of a combination of pharmacologically active agents characterized in that said medicament is adapted to treat presbyopia.

Therefore, the difference of both patents (the European patent EP3031457 as the Spanish patent ES2725002) with the present invention, is that this patent application describes a specific ophthalmic composition to diminish or eliminate the symptoms of presbyopia where the combination of pilocarpine, brimonidine, oxymetazoline, hyaluronic acid, bromfenac and/or at least one vasoconstrictor, is in a single dose of low concentration, significantly diminishing or eliminating the side effects.

Likewise, patent EP3031457 mentions examples that are not specific for presbyopia, since it mentions:

“A second eye drop formulation materializing the present invention was prepared by incorporating 0.1% by weight of brimonidine and 0.25% by weight of pilocarpine in an otherwise conventional eye drop formulation. Brimonidine is classified as a sympathetic agonist; pilocarpine, as noted above, is considered a parasympathetic agonist. The second eye drop formulation was evaluated in the three patients noted above. In each case, administration of the second formulation of eye drops produced nearly identical effects to those of the first formulation of eye drops, above, where a sixty-three-year-old patient presented as emmetropic (not requiring glasses for functional distance vision). The patient's vision was assessed, the first eye drop formulation was administered, and the patient's vision was reassessed. Twenty minutes after administration, the patient's unaided distance vision in each eye improved by one line on the Snellen chart, from 6/6 to 6/5. Refraction did not change. The patient's unaided reading vision improved from N12 to N4.5 at a reading distance of one-third of a meter. The patient's night vision improved qualitatively, as the patient noticed fewer halos and glare, and quantitatively, from 6/6 to 6/5 in dim light conditions. These effects were maintained for at least two hours and some for at least four hours.”

Therefore, it is only an anecdotal hypothesis of a single patient, where a clinical study in a group of patients is not established and where specific results to mitigate presbyopia are not shown.

Patent MX963060 applied for in the year 1996 with the title “Compositions for the treatment of glaucoma comprising pilocarpine and a beta-blocker” refers to an ophthalmic composition for controlling glaucoma and/or ocular hypertension, comprising 0.1 to 1.0% w/v of a beta-blocker such as betaxolol, 0.25 to 10.0% w/v of pilocarpine, 0.05 to 10.0% w/v of an ion exchange resin in poly(styrene-divinylbenzene)-sulfonic acid, and 0.01 to 5.0% w/v of a polyanionic polymer is carbomer 934P; therefore, the difference with the present invention is that this composition is for treating glaucoma and not presbyopia, and although it mentions pilocarpine, its use is different.

International Patent Application WO2015092087 dated Jun. 25, 2015, with the title “Ophthalmic composition for the correction of presbyopia” refers to an ophthalmic composition for the correction of presbyopia which comprises the following: Pilocarpine 2%, diluted to 1% with sterile balanced salt solution (BSS), yielding pilocarpine 1% (2.5 ml)+bromfenac, 1.8 mg bromfenac (2.5 ml)+sterile balanced salt solution (BSS) (2.5 ml), yielding in total an eye drop of 7.5 ml; therefore, although this international application mentions a composition for treating presbyopia with pilocarpine, the composition does not include brimonidine or the other components of the present invention, such as oxymetazoline, among others, so it turns out to be totally different.

The literature paper with the title “Clinical outcomes of combined versus separate carbachol and brimonidine drops to correct presbyopia” Almamoun Abdelkader1* and Herbert E. Kaufman2, 11 Nov. 2016, relates to testing and comparing in a masked fashion the efficacy of using a parasympathomimetic drug. (3% carbachol) and an alpha-2 agonist (0.2% brimonidine) in combined and separate forms to create optically beneficial myosis to pharmacologically improve vision in presbyopia. Instead, the present invention, is that said composition described in said article mentions only brominidine with carbachol, which said composition does not include pilocarpine, wherein said ingredient makes it more effective.

Patent Application MX/a/2015/000806 filed in year 2015, with the title “Ophthalmic formulation and method for mitigating presbyopia” relates to an ophthalmic formulation comprising an effective amount of pilocarpine or a pharmaceutically acceptable pharmaceutically effective salt thereof, and one or more a1-adrenergic agonists or agonists are selected from the group consisting of phenylephrine, phenylpropanolamine, etilefrine, oxymetazoline, xylometazoline, tramazoline, and a pharmaceutically acceptable salt thereof, wherein preferably it is phenylephrine, which is present in an amount of 0.5% to 3% by weight, or preferably in an amount of 0.7% to 2.2%. It further comprises an antihistamine which is pheniramine or a pharmaceutically acceptable salt thereof, which is present in an amount of 0.03% to 0.09% by weight and a non-steroidal anti-inflammatory drug which is selected from nepafenac and meloxicam. The ophthalmic formulation is used to mitigate, reduce or treat presbyopia. While this patent mentions pilocarpine, it does not include brimonidine or the other components of the present invention, such as bromfenac. In addition, the effectiveness of said ophthalmic formulation is different from that described in the present invention.

Patent MX351230 applied for in 2013 with the title “Ophthalmic formulation and method for mitigating presbyopia” relates to an ophthalmic formulation comprising an effective amount of pilocarpine or a pharmaceutically acceptable salt thereof, naphazoline or a pharmaceutically acceptable salt thereof, and a pharmaceutically acceptable excipient, wherein phenylephrine is present in an amount of 0.55 to 3% by weight, pilocarpine is present in an amount of 0.1% to 0.7% by weight, and naphazoline is present in an amount of 0.001% to 0.020% by weight. The ophthalmic formulation may enable the treatment of conditions that adversely affect a patient's visual acuity, including presbyopia which may treat it or mitigate its symptoms. Consequently, it is concluded that, although this patent mentions pilocarpine, it does not include brimonidine or the other components of the present invention, such as oxymetazoline and bromfenac, so that the effectiveness of said ophthalmic formulation is different.

Patent MX357635 applied for in 2012 with the title “Compositions and methods for the treatment of presbyopia, mild hyperopia and irregular astigmatism” relates to the use of a composition comprising a therapeutically effective amount of pilocarpine 1% w/w and oxymetazoline in an amount of 0.012%, 0.025% and 0.125% for the preparation of a medicament for the treatment of presbyopia, wherein the composition is administrable in the eye of the subject.

Patent MX357635 an alpha agonist, which has an imidazoline group or a nonsteroidal anti-inflammatory agent (NSAID) that has selectivity against COX-2 in combination with a cholinergic agent, such as pilocarpine, has been found to act synergistically to improve the accommodative and focusing ability of the eye while minimizing the side effects of each compound.

Therefore, the difference with the present invention is that this composition does not comprise brimonidine or hyaluronic acid and although the composition minimizes the side effects of each compound, it does not eliminate the side effects, thus having better synergy and better tolerance at lower dosage.

Patent Application MX/a/2019/053150 applied for in 2019, with the title “Ophthalmic pharmaceutical compositions and uses relating thereto” relates to an ophthalmic pharmaceutical composition comprising pilocarpine or a pharmaceutically acceptable salt thereof at a concentration of 0.01% (w/w or w/v) to 0.4% (w/w or w/v) and a pharmaceutically acceptable carrier, wherein the pilocarpine salt is pilocarpine hydrochloride or pilocarpine nitrate. Further the composition comprises diclofenac or a pharmaceutically acceptable salt thereof at a concentration of 0.001% (w/w or w/v) to 0.090% (w/w or w/v), or ketorolac or a pharmaceutically acceptable salt thereof at a concentration of 0.01% (w/w or w/v) to 0.60% (w/w or w/v) wherein the salt of diclofenac is diclofenac sodium, or wherein the salt of ketorolac is ketorolac tromethamine. It further comprises a lubricating agent such as hyaluronic acid or a pharmaceutically acceptable salt thereof, cellulose or derivative thereof, sodium carboxymethylcellulose, hydroxyethylcellulose, hydroxypropylmethylcellulose, methylcellulose, dextran, gelatin, a polyol, glycerin, polyethylene glycol 300, polyethylene glycol 400, polysorbate, propylene glycol, polyvinyl alcohol, povidone, or mixtures thereof; wherein the lubricating agent is sodium hyaluronate, or hydroxypropyl methylcellulose, or mixtures thereof. The sodium hyaluronate is present at a concentration of 0.01% (w/w or w/v) to 0.9% (w/w or w/v), and/or wherein the hydroxypropyl methylcellulose is present at a concentration of 0.1% (w/w or w/v) to 2.0% (w/w or w/v).

Therefore, the difference with the present invention is that the patent application MX/a/2019/053150 does not comprise brimonidine or oxymetazoline, so the present invention mitigates presbyopia having a greater effect in lower dose and better tolerance without having side effects.

There are several efforts to mitigate the symptoms of presbyopia using drugs targeting the iris sphincter and dilator in people with presbyopia. In 1990 Rosenfield documented the use of phenylephrine and thymoxamine (an alpha-antagonist) achieving 1.5 D of accommodative amplitude lasting only for two hours. (Rosenfield M. The Influence of Alpha-adrenergic-agents on tonic accommodation. Current eye research, vol. 9, 3, 1990, pp 267-272).

Benozzi reported a combination of pilocarpine 1% to 2% combined with diclofenac to treat presbyopia in individuals over a 5-year period. At such a dose 20% of patients experienced burning eyes, ocular discomfort immediately after instillation of drops. Discontinued treatment due to medication intolerance, 4% preferred the use of glasses. (Jorge Benozzi Presbyopia: a New Potential Pharmacological Treatment MEHDI Ophthalmology Journal 2012; Vol. 1, No. 1.

In 2015 Abdelkader reported a combination of carbachol 2.25% with brominidine 0.2% (commercial concentration) used monocularly in individuals with presbyopia and compared to placebo achieved significant improvement in near visual acuity compared to placebo. 10% of individuals reported acute headache. (Abdelbaker A. Improved Presbyopic Vision with Myotics Eye Contact Lens. 2015 September; 41(5): 323-327) A year later he published his results of 10 individuals with presbyopia using the same compound with 3% carbachol and compared to brimonidine 0.2% and 3% carbachol alone.

Renna published in 2016 a composition including pilocarpine 0.247%, phenylephrine 0.78%, polyethyleneglycol 0.09%, nepafenac 0.023%, pheniramine 0.034% and naphazoline 0.003% instilled binocularly in 14 subjects with presbyopia. The results showed 2 to 3 lines of Uncorrected Near Visual Acuity (NCVA). No patient had loss of far vision lines in each eye and binocularly. Renna A, Vejarano L F, De la Cruz E, Alió J L. “Pharmacological treatment of presbyopia by novel binocularly instilled eye drops: a pilot study”. Ophthalmol Ther. 2016, 5(1): 63-73.

In 2018 Vargas, from the same study group published a series of 117 patients with presbyopia using the same formulation where they achieve significant improvement of CNCAVA in 92.3% of patients for 2 hours after instillation of the eye drops. 14 patients reported headache, 1 patient was intolerant to the preparation. Vargas V, Vejarano F, Alió J L. Near Vision Improvement with the Use of a New Topical Compound for Presbyopia Correction: A Prospective, Consecutive Interventional Non-Comparative Clinical Study. Ophthalmol Ther. 2019; 8(1):31-39. doi:10.1007/s40123-018-0154-6.

The problem with the above formulations is that when pilocarpine alone is used it causes headache, irritation, and intolerance to the components.

Therefore, the present invention protects an ophthalmic pharmaceutical composition of pilocarpine, brimonidine and oxymetazoline in a single dose of low concentration with improved tolerability in combination with hyaluronic acid, bromfenac and/or at least one vasoconstrictor, preventing inflammation, dryness, headache and eliminating eye redness.

### OBJECT OF THE INVENTION

To obtain a new therapeutic option of ophthalmic pharmaceutical composition comprising the synergistic combination of: one or more cholinergic agonist agents, preferably selected from pilocarpine or its racemic forms or its pharmaceutically acceptable salts, one or more alpha adrenergic 1 agonist agents, preferably selected from oxymetazoline or its racemic forms or its pharmaceutically acceptable salts, one or more alpha adrenergic 2 agonist agents, preferably selected from brimonidine or its racemic forms or its pharmaceutically acceptable salts, one or more glycosaminoglucuronan agents, preferably selected from hyaluronic acid or its racemic forms or its pharmaceutically acceptable salts and one or more non-steroidal anti-inflammatory agents, preferably selected from bromfenac or its racemic forms or its pharmaceutically acceptable salts and/or one or more vasoconstrictor agents or its racemic forms or its pharmaceutically acceptable salts; plus pharmaceutically acceptable vehicles, excipients or adjuvants, formulated in a pharmaceutical form of solution for ophthalmic administration. Said combination is indicated for the prevention, control, and eradication of the symptoms of presbyopia, to increase the depth of focus in the eyes of patients suffering from presbyopia and to improve near vision ability.

The pharmaceutical composition comprising the present combination of compounds is useful in decreasing or eliminating side effects such as: conjunctival irritation, eye pain, dryness, decreased distance vision, redness of the eye, eye irritation, headache, posterior synechiae due to chronic use of medications. In addition, it helps to increase the depth of focus in the eyes of patients suffering from presbyopia to improve near vision ability.

The ophthalmologic composition produces a greater therapeutic effect when administered in a single dose, as opposed to when they are administered independently, generating benefits; lower doses administered, greater therapeutic effect and the decrease or elimination of adverse effects.

### DETAILED DESCRIPTION OF THE INVENTION

The present invention relates to a novel ophthalmic delivery composition comprising a synergistic combination of one or more cholinergic agonist agents or their racemic forms or their pharmaceutically acceptable salts, more specifically selected from pilocarpine or its hydrochloride or nitrate salts; one or more alpha adrenergic agonist agents 1 or their racemic forms or their pharmaceutically acceptable salts, more specifically selected from oxymetazoline or its hydrochloride salts; one or more alpha adrenergic agonist agents 2 or their racemic forms or their pharmaceutically acceptable salts, more specifically selected from brimonidine or its tartrate salts; at least one or more glycosaminoglucuronan agents or their racemic forms or their pharmaceutically acceptable salts, more specifically selected from hyaluronic acid or its sodium salt; at least one vasoconstrictor or their racemic forms or their pharmaceutically acceptable salts, selected from: epinephrine, norepinephrine, levonordephrine, phenylephrine and/or at least one non-steroidal anti-inflammatory drug or its racemic forms or its pharmaceutically acceptable salts selected from acetylsalicylic acid, lysine clonixinate, clonixin, benorilate, diflunisal, salicylamide, etersalate, salsalate, salicylic acid, acemetacin, glucamethazine, indomethacin, proglumetacin, oxamethacin, sulindacin, tolmetin, difenpyramide, aceclofenac, bromfenac, diclofenac, etodolac, fenthiazac, ketorolac, bufexamac, lonazolac, alclofenac, zomepiraco, droxicam, meloxicam, piroxicam, tenoxicam, oxaprocin, phenylbutazone, mofebutazone, oxyphenbutazone, clofezone, kebuzone, metamizole, feprazone, nifenazone, suxibuzone, aminophenazone, butybufen, fenoprofen, fenbufen, flurbiprofen, benoxaprofen, suprofen, ibuprofen, ibuproxam, ketoprofen, dexketoprofen, pirprofen, indoprofen, naproxen, oxaprozin, tiaprofen, dexibuprofen, fenoprofen, flunoxaprofen, alminoprofen, meclofenamic acid, mefenamic acid, flufenamic acid, tolfenamic acid, niflumic acid, etofenamate, celecoxib, rofecoxib, parecoxib, valdecoxib, etoricoxib, paracetamol; wherein said composition further comprises at least one pharmaceutically acceptable vehicle, excipient or adjuvant to obtain said ophthalmological composition, formulated in a liquid or semi-solid pharmaceutical form, in a single dose of low concentration, with an increased tolerance, which prevents and/or controls and/or eradicates the side effects of pilocarpine such as eye redness, eye irritation, headache and eye dryness, and increases the depth of focus in the eyes of patients suffering from presbyopia to improve near vision ability.

The herein synergistic pharmaceutical composition produces a greater therapeutic effect when they are applied together in a single dose of administration with the standard treatment for an eye-related disease as opposed to when they are administered independently, generating benefits; lower dose administered, greater therapeutic effect and free of adverse effects.

The vehicle is selected from at least one compound selected from purified water or sterile water and the excipients and/or adjuvants are selected from at least the following groups of compounds: at least one isotonizing compound selected from sodium chloride, sodium sulfite, potassium nitrate or potassium chloride; at least one antioxidant compound selected from EDTA disodium salt, sodium metabisulfite or ascorbic acid; at least one pH-regulating compound and/or buffer selected from boric/borate; at least one surfactant selected from polysorbate 20, polysorbate 80, triton WR, lecithins or polyethylene-polypropylene glycol; at least one viscosifier selected from hydroxypropyl methylcellulose, ethyl cellulose, hydrocyethyl cellulose, sodium carboxymethyl cellulose, polyvinyl alcohol, polyvinyl pyrrolidone, dextran 70, carbopol, polyacrylamide or sodium chondroitin sulfate.

One embodiment of pharmaceutical form of the present ophthalmic composition is liquid or semi-solid, preferably in liquid form in eye drop type solution, wherein the vehicle is selected from purified water, pharmaceutical grade water or sterile water.

The herein invention comprises: a) pilocarpine, a parasympathetic mimetic drug binding to m3 muscarinic receptors. said drug is mainly used for the treatment of narrow-angle glaucoma in concentrations ranging from 1% to 4% causing miosis, wherein contraction of the ciliary body increases uveo-scleral outflow and then decreases intraocular pressure. side effects produced by pilocarpine include ciliary spasm, myopization, red eyes, eyebrow pain and posterior iris synechiae from chronic use; b) oxymetazoline, is an alpha 1 adrenergic agonist drug used primarily as a vasoconstrictor for the treatment of red eye, and to have a dilating effect on the pupil; c) brominidine, is an alpha-2 adrenergic agonist drug used for the treatment of glaucoma at concentrations of 0.15% to 2%. It blocks the alpha 2 receptors of the iris dilator muscle, inhibiting its function, and then introducing miosis. This side effect has been used to treat night glare and halos experienced by patients after excimer laser procedures; d) hyaluronic acid or its sodium salt (HA) is a linear polymer, of high molecular weight and natural origin; its monomeric unit is constituted by the disaccharide composed of N-acetyl-ß-D-glucosamine and ß-D-glucuronic acid. It is a vital component of ocular physiology that provides the viscosity required by the vitreous humor and keeps the corneal epithelium hydrated. It has the ability to be one of the eye's natural lubricants and has been used to develop ophthalmic applications such as replacement of natural vitreous humor, protection of the corneal endothelium against mechanical trauma during surgery and to mimic natural tears in the treatment of dry eye. It is a natural polysaccharide compound (glycosaminoglycan) found in significant amounts in synovial fluid and vitreous humor. Hyaluronic acid is commonly used as a tear replacement in the treatment of dry eyes (xerophthalmia), it is also used as a surgical support in cataract extraction (intra and extracapsular), intraocular lens implantation (IOL), corneal transplantation, glaucoma filtration, retinal docking surgery, maintaining a deep compartment to allow for more efficient manipulation, causing less trauma to the endothelium and other surrounding tissues, preventing postoperative flat chamber formation and creating a clear field of view for intraoperative and postoperative retinal inspection and photocoagulation; e) bromfenac ophthalmic, is a drug that belongs to a class of medications known as non-steroidal anti-inflammatory drugs (NSAIDs), which works to stop the release of certain natural substances that cause pain or swelling. It is used to treat eye inflammation and redness (swelling) and pain that occurs after cataract surgery.

More preferably, the ophthalmic composition comprises pilocarpine in an amount of between 0.05 to 0.50%; sodium hyaluronate/oxymetazoline in an amount of between 1.00 to 2.5 mg/0.10 mg to 0.5; brimonidine in an amount of between 0.01% to 0.1%; bromfenac in an amount of between 0.10 to 0.5 mg and/or at least one vasoconstrictor and at least one pharmaceutically acceptable excipient or vehicle.

The following more particularly describes realizations of the present invention by means of a clinical study.

Clinical Trial

A composition was prepared where patients with presbyopia symptoms were selected. This study was approved by the ethics committee of Optall Vision Eye Center in Mexico City. All patients signed and gave their consent to participate in such study.

For the study, patients with presbyopia, with a corrected visual acuity of 20/25 or better, were included. The diagnosis of presbyopia was determined without correcting near vision acuity was equal to or less than 3 on the Jaeger scale on the Rosenbaum chart improving at least 2 on the Jaeger scale with the use of at least +1.00 D. Age, gender, ocular dominance, uncorrected visual acuity (UCVA), corrected near visual acuity (BNCVA) intraocular pressure with Goldmann applanation tonometer (IOP), pupil diameter under photopic and scotopic conditions (PD), slit lamp biomicroscopic examination and a fundus examination were recorded.

The ophthalmic composition comprised a single low concentration dose of pilocarpine, brimonidine, oxymetazoline/sodium hyaluronate, bromfenac and/or at least one vasoconstrictor and at least one pharmaceutically acceptable vehicle. From said combination, one drop of the synergistic mixture was applied and instilled in the nondominant eye and one drop of pilocarpine or brimonidine dilution was alternately instilled in the dominant eye of consecutive patients. After one hour, uncorrected visual acuity (UCVA), corrected visual acuity (BCVA), uncorrected near visual acuity (UNVA), corrected near visual acuity (BCNVA), intraocular pressure (IOP), pupil diameter (PD), visual analog pain scale (VAS) was measured from 0 to 10. Hyperemia was measured from 1 to 4.

Statistical analyses were performed with SPSS using Pearson's and Spearman's correlation analysis.

The type of modern Jaeger eye chart scale used generally ranges from J10 (approximately 14 points for Times New Roman typeface) to J1 (approximately 3 points, Times New Roman typeface). The chart moves back and forth until you can read a certain font size.

The results of the descriptive statistics, in which eleven patients were recruited to participate in the study, 7 males and 4 females. In 6 patients the dominant eye was the right eye and in 5 patients the dominant eye was the left eye. Mean age was 49.27±1.84 SD range (44-56 years). Visual acuity improvement (BCVA) was 20/20 in all eyes. In the Jaeger scale it was a binocular measurement and individually for each eye. Table 1 mentions that the binocular Jaeger scale data was 5.82±−1.04 SD range (3-7).

On the Jaeger scale for the right eye it was 5.91±1.514 SD range (3-8). On the Jaeger scale for the left eye was 5.64±−1.502 SD range (3-7). Intraocular pressure OD was 13 mmHg±−2.145 SD range (10-16 mmHg). OD photopic pupil diameter was 3.73 mm±−0.46 SD range (3-4 mm). OS photopic pupil diameter was 3.73 mm±0.46 SD (3-4 mm). Scotopic pupil diameter OD was 4.63 mm±−0.636 SD range (3.5-5 mm).

In Table No. 1 (Lines gained in the Jaeger reading primer. Basal, One hour after instillation, synergistic combination; pilocarpine or brimonidine added together, pilocarpine and brimonidine alone) mentions the synergistic combination of the present invention where the Jaeger scale was 2.09±1.446 SD range (1-5) (p 0.0001). Jaeger in the dominant eye with pilocarpine or brimonidine was 3.82±−0.601 SD range (1-6) (p. 0.032). Jaeger scale instilled with pilocarpine was 4.20±−1.483 SD range (2-6) (p. 102). Jaeger in the eyes instilled with Brimonidine was 3.50±−1.761 range (1-5) (p 0.250).

TABLE NO. 1

| DRUG | JAEGER- 2 | SD | RANGE | P |
|---|---|---|---|---|
| Baseline | 5.82 | 1.04 | | |
| 1 h after | 2.45 | 1.36 | 1 a 4 | 0.003 |
| Synergistic | 2.09 | 1.44 | 1 a 5 | 0.0001 |
| mixture | | | | |
| Pilocarpine or | 3.82 | 1.6 | 1 a 6 | 0.032 |
| Brimonidine | | | | |
| Pilocarpine | 4.2 | 1.48 | 2 a 6 | 0.102 |
| Brimonidine | 3.5 | 1.76 | 1 a 5 | 0.25 |

In Table No. 2 (Lines gained in the reading primer with Jaeger's notation, synergistic combination), it is mentioned that the lines gained with the synergistic combination in the non-dominant eye was 3.73±1.42 SD range (2-6). Lines gained in the dominant eye with pilocarpine or brimonidine was 2±−1.89 SD range (0-6). Lines gained in eyes with pilocarpine was 1.60±−089 SD range (1-3). Lines gained in eyes with brimonidine was 2.33±−2.50 SD range (0-6). Intraocular pressure OD was 13 mmHg±−2.145 SD range (10-16). OS intraocular pressure was 13 mmHg±−2.145 SD range (10-16).

TABLE NO. 2

| DRUG | WINNED LINES | SD | RANGE |
|---|---|---|---|
| Synergistic | 3.73 | 1.42 | 2 a 6 |
| combination | | | |
| Pilocarpine or | 2 | 1.89 | 0 a 6 |
| Brimonidine | | | |
| Pilocarpine | 1.6 | 0.89 | 1 a 3 |
| Brimonidine | 2.33 | 2.5 | 0 a 6 |

In Table No. 3 (Pupillary size in light (photopic) and dark (scotopic) conditions; PD Pupillary diameter, synergistic combination) mentions that the OS scotopic pupillary diameter was 4.63 mm±−0.636 SD range (3.5-5 mm). The synergistic combination was instilled in the non-dominant eye of all participants. Dilutions of pilocarpine and brimonidine were alternately instilled in the dominant eye of all participants. In Table No. 1 it mentions that one hour after binocular drops on the Jaeger scale was 2.45±1.36 SD range (1-4) (p 0.003).

In addition, Table No. 3 mentions the photopic pupillary diameter of the synergistic mixture in the non-dominant eye was 2.63 mm±−0.63 SD range (2-4) (p. 0.039). The photopic pupillary diameter of pilocarpine or brimonidine in the dominant eye was 3.13 mm±−0.63 SD range (2-4) (p. 0.059). Scotopic pupillary diameter in the non-dominant eye was 2.68 mm±0.60 SD range (2-4) (p 0.042). Scotopic pupillary diameter with pilocarpine, brimonidine in the dominant eye was 2.90 mm±0.66 SD range (2-4) (p 0.061).

The results showed that pain in the combination of pilocarpine, brominidine in the non-dominant eye was at AVS 0. Pain in the dominant eye with pilocarpine or brimonidine at one AVS was reported by two patients at grade 2 and 3 respectively.

TABLE NO. 3

| DRUG | PHOTOPIC PD | SD | RANGE | P |
|---|---|---|---|---|
| Baseline | 3.73 | 0.46 | 3 a 4 | |
| Synergistic | 2.63 | 0.63 | 2 a 4 | 0.039 |
| combination | | | | |
| Pilocarpine or | 3.13 | 0.63 | 2 a 4 | 0.059 |
| Brimonidine | | | | |

| | Photopic PD | | | |
|---|---|---|---|---|
| Baseline | 4.63 | 0.63 | 3.5 a 5 | |
| Synergistic | 2.68 | 2.6 | 2 a 4 | 0.042 |
| combination | | | | |
| Pilocarpine or | 2.9 | 0.66 | 2 a 4 | 0.061 |
| Brimonidine | | | | |

The hyperemia reading in the present synergistic combination in the nondominant eye was 1 in eye 1 of 11 (9%). Hyperemia in the dominant eye with pilocarpine or brimonidine was present in 4 patients, 3 with grade 1 hyperemia and 1 with grade 2 hyperemia (36%). BCVA was maintained 20/20 in all patients, no patient reported blurred distance vision.

Thus, it is observed, with the results obtained and unexpectedly and surprisingly, that the combination of the drugs in a single dose of low concentration showed an improved synergistic effect on near vision as measured with the Rosembaum Chart with Jaeger notation, compared to the administration of pilocarpine and brimonidine if instilled separately after one hour in the non-dominant eye of patients with presbyopia. It is feasible that the present invention be formulated and developed in a pharmaceutical composition to be administered ophthalmically, which implies, lower dosage, higher therapeutic potency, and reduced risk of adverse events, for the treatment of presbyopia.

### EXAMPLES

The following is a non-limiting description, by way of example, of some pharmaceutical compositions:

### Example 1: Composition for Ophthalmic Administration

At least one cholinergic agonist agent
At least one alpha adrenergic 1 agonist agent
At least one alpha 2-adrenergic agonist agent
At least one glycosaminoglucuronan agent
At least one non-steroidal anti-inflammatory
agent and/or at least one vasoconstrictive agent
At least one pharmaceutically acceptable
excipient and/or vehicle

The herein invention can be represented in other specific forms without departing from its spirit or essential features, such as parenteral, muscular, intravenous injections; tablets, hard or soft gelatin capsules, micro pellets, caplets, powder for reconstitution, and can present different modified release systems such as: controlled release, sustained release, pulsatile release. The described modalities will be considered in all aspects, only as illustrative and not as restrictive. The concentration of the present pharmaceutical combination may vary according to the necessary adjustments according to the results in the clinical study.

Comprehensively, the present invention provides the following advantages:
1. Increases the depth of focus in the eyes of patients suffering from presbyopia,
2. Improves near vision ability.

Finally,
1. The results establish that the combination is useful for the treatment of presbyopia.

1. A pharmaceutical composition for ophthalmic administration for use in the prevention, control, and treatment of the symptoms of presbyopia and to improve near vision ability and increase the depth of focus in the patient's eyes, comprising the synergistic combination of:
i. One or more cholinergic agonist agents or their racemic forms or pharmaceutically acceptable salts thereof, preferably selected from pilocarpine
ii. One or more alpha 1-adrenergic agonist agents or their racemic forms or their pharmaceutically acceptable salts, preferably selected from oxymetazoline
ii. One or more alpha 2-adrenergic agonist agents or their racemic forms or their pharmaceutically acceptable salts, preferably selected from brimonidine
iv. One or more glycosaminoglucuronan agents or their racemic forms or their pharmaceutically acceptable salts, selected preferably from hyaluronic acid
v. One or more non-steroidal anti-inflammatory agents or their racemic forms or pharmaceutically acceptable salts thereof, selected from bromfenac
vi. One or more vasoconstrictor agents or their racemic forms or their pharmaceutically acceptable salts, preferably selected from oxymetazoline, phenylephrine, tetrahydrozoline and naphazoline
vii. A pharmaceutically acceptable vehicle, adjuvant, or excipient.

2. A pharmaceutical composition for ophthalmic administration according to claim 1, characterized in that pilocarpine is selected from the hydrochloride salt, oxymetazolone is selected from the hydrochloride salt, brimonidine is selected from the tartrate salt, hyaluronic acid is selected from the sodium salt, bromfenac is selected from the sodium salt, and vasoconstrictor is selected from the chlorhidrate salt.

3. A pharmaceutical composition for ophthalmic administration according to claim 1, characterized in that pilocarpine, brimonidine and oxymetazoline are present in a single low concentration dose comprising pilocarpine in an amount of 0.05% to 0.5%; brimonidine in an amount of 0.01% to 0.1% and oxymetazoline in an amount of 1.0 mg to 0.5 mg, hyaluronic acid is in an amount of 0.1 mg to 2.5 mg.

4. A pharmaceutical composition for ophthalmic administration according to claim 1, characterized in that pilocarpine and brimonidine are diluted in a 0.5% to 1.0% saline solution.

5. A pharmaceutical composition for ophthalmic administration according to claim 1, for use in addition, in eliminating the side effects of pilocarpine such as eye redness, headache and dryness of the eye and there is increased tolerance in patients suffering from presbyopia.
